(19) United States
(12) Patent Application Publication
(10) Pub. No.: US 2010/0269830 A1
(43) Pub. Date: Oct. 28, 2010

(54) Fluid Removing Apparatus for Respiratory Tract

(75) Inventors: James Layer, Crystal Lake, IL (US); Timothy S. Moran, Rockford, IL (US)

Correspondence Address: MILLER, MATTHIAS & HULL, ONE NORTH FRANKLIN STREET, SUITE 2350, CHICAGO, IL, 60606, US

(73) Assignee: Sage Products, Inc., Cary, IL (US)

(21) Appl. No.: 12/429,788

(22) Filed: Apr. 24, 2009

(51) Int. Cl. A61M 1/00; A61M 16/04

(52) U.S. Cl. 128/205.27; 128/207.14

(57) ABSTRACT

A medical apparatus for removing fluid accumulated along a respiratory tract of a patient, and particularly around an endotracheal tube, is disclosed. The disclosed apparatus may include a tube defining a lumen extending between a distal fluid port and a proximal fluid port, wherein the tube forms a self-supported, three-dimensional structure. The three-dimensional structure may include a distal coil adapted to be slidably and rotatably mounted on the exterior surface of the endotracheal tube. The three-dimensional structure may also include a proximal coil.

### FIELD OF THE DISCLOSURE

The present disclosure generally relates to medical devices and, more particularly, relates to a medical device for removing fluid accumulated along a respiratory tract and around an endotracheal tube. The disclosed apparatus may include a tube defining a lumen extending between a distal fluid port and a proximal fluid port, wherein the tube forms a self-supported, three-dimensional structure. The three-dimensional structure may include a distal coil adapted to be slidably mounted on the exterior surface of the endotracheal tube.

### BACKGROUND OF THE DISCLOSURE

Endotracheal tubes (also called ET tubes or ETTs) for keeping airways open in a respiratory tract of a patient are well known in the art. In use, the tube is inserted into the mouth of the patient, past the epiglottis and into the trachea in order to ensure that the airway is not closed off and that air is able to reach the lungs. The endotracheal tube is regarded as a reliable method for protecting the airway of a patient, and are used in many situations including general anesthesia, intensive care and emergency medicine for airway management and mechanical ventilation.

There are a variety of different types of currently available endotracheal tubes. For example, endotracheal tubes may be inserted in the respiratory tract either orally or nasally. Further, some endotracheal tubes may include an inflatable cuff at a distal end to anchor the tube in the trachea and prevent fluid leak that may interfere with the medical procedure that is performed on the patient. A cuffed endotracheal tube typically includes a small inflation lumen to selectively inflate the cuff after the endotracheal tube is properly positioned and to deflate the cuff before the endotracheal tube is removed from the patient's respiratory tract.

Endotracheal tubes may have a straight design, or they may include pre-formed bend to allow accurate placement of the tubes without having to carefully measure its length below the vocal cords, in which case the tubes are generally referred to as RAE tubes. Endotracheal tubes range in size from 2-10.5 mm in internal diameter (ID). Different sizes are chosen based on the body size of the patient with the smaller sizes being used mostly for pediatric and neonatal patients. Endotracheal tubes larger than 6 mm ID tend to have an inflatable cuff, although recently nearly all tubes can be equipped with a cuff.

Besides the traditional double-lumen design in cuffed endotracheal tubes, triple-lumen endotracheal tubes have been developed for intra-thoracic surgery. These tubes allow single-lung ventilation whereas the other lung can be collapsed to make surgery proceed easier. At the end of the surgery, the other lung may be reinflated through the endotracheal tube.

One typical problem associated with an endotracheal tube is the accumulation of fluid between the tube and the respiratory tract. This is particularly true with cuffed tubes in that the cuff forms a relatively consistent seal with the interior of the trachea and thus fluid tends to accumulate above the cuff. The accumulated fluid may include body fluid from the patient, such as saliva or mucus secreted from the respiratory tract. Moreover, the accumulated fluid may also include residues of fluids introduced during various medical or hygiene procedures, such as dental composition, oral cleaning compositions, and the like. The accumulated fluid may include harmful microorganisms that, when leaked into the lungs, may cause adverse medical effect such as infection. This is especially problematic in that the cuff and distal portion of the endotracheal tube are typically inserted below the epiglottis thereby penetrating a protective layer of the body not normally exposed to such fluid and bacteria. The infection that generally develops as a result of leakage of pathogenic organisms when an endotracheal tube is in place is called a VAP (ventilator associated pneumonia), which is one of the most commonly acquired infections in the Intensive Care Units of hospitals with over 250,000 VAP's acquired each year in the U.S. and mortality rates ranges from 24% to 76%. As a result, timely and effective removal of the accumulated fluid is desirable in the use of endotracheal tubes.

To that end, another type of triple-lumen endotracheal tube has been developed which includes an inner tube disposed within an outer tube, as well as an inflation tube to inflate or deflate the cuff. The inner tube extends between a proximal opening connected to a suction device and a distal opening positioned immediately above the inflatable cuff. In use, fluid accumulated around the endotracheal tube and above the inflatable cuff may be suctioned out through the inner tube, while the space between the inner and outer tubes maintains an open airway along the respiratory tract.

This triple-lumen design, although effective in removing the accumulated fluid, has several drawbacks in its application due to the fact that the inner tube is integrated with the outer tube. For example, when the inner tube gets clogged, the suction device connected to the inner tube needs to be removed and replaced with an pressurized air source to blow open the clogged inner tube, which may cause additional discomfort to the patient. A possible alternative solution is to have the entire assembly removed from the patient and replaced with an unclogged assembly, which is not only time and labor-consuming, but also causes significant discomfort to the patient. Further, in emergency situations, the majority of emergency medical technicians, ambulances and paramedics only stock and employ traditional, double-lumen cuffed endotracheal tubes. Accordingly, if a physician wishes to use the triple-lumen type once the patient reaches the hospital, the already inserted double-lumen endotracheal tube needs to first be removed, causing unnecessary discomfort and trauma to the patient, as well as waste and expense to the medical service providers.

Hence, there is a need for a medical apparatus that effectively removes fluid accumulated subglotticly along a respiratory tract, particularly around an endotracheal tube inserted therein. Moreover, there is a need for a fluid-removing apparatus that can be conveniently and securely mounted along the exterior surface of an endotracheal tube. Finally, there is need for a robust fluid-removing apparatus that can slide along an endotracheal tube to remove fluid accumulated between the endotracheal tube and a respiratory tract without causing trauma to the respiratory tract.

### SUMMARY OF THE DISCLOSURE

This disclosure is generally directed toward a medical apparatus for removing fluid accumulated along a respiratory tract. More specifically, the disclosed apparatus may remove fluid accumulated between the respiratory tract and an endotracheal tube inserted therein.

In a general embodiment, the disclosed medical apparatus may include a fluid-removing tube having a distal fluid port and a proximal fluid port thereon. The fluid-removing tube also defines a lumen extending between the distal fluid port and the proximal fluid port. In operation, the fluid-removing tube is positioned so that the distal fluid port is in communication with the accumulated fluid. The accumulated fluid may then be removed from the respiratory tract by applying a pressure differential between the proximal fluid port and the distal fluid port of the disclosed apparatus.

In some embodiments, the fluid-removing tube may have an open distal end, in which case the distal fluid port may be the distal opening of the fluid-removing tube. In other embodiments, the distal end of the fluid-removing tube may be closed, in which case the distal fluid port may be provided as a transverse opening on the tubular wall close to the distal end of the fluid-removing tube.

Similarly, the fluid-removing tube may have an open proximal end, in which case the proximal fluid port may be the proximal opening of the fluid-removing tube. Alternatively, the proximal end of the fluid-removing tube may be closed, in which case the proximal fluid port may be provided as a transverse opening on the tubular wall close to the proximal end of the fluid-removing tube.

The tube may have enough rigidity to form a self-supported, three-dimensional structure. For example, the tubular wall may be manufactured from a relatively rigid material. Alternatively, the tube may include a relatively rigid support element, such as a metal wire, to form the three-dimensional structure. The three-dimensional structure may include a distal coil adapted to be slidably mounted along the exterior surface of the endotracheal tube. The three-dimensional structural may also optionally include a proximal coil, which may also be slidably mounted along the exterior surface of the endotracheal tube. In one embodiment, the pitch of the distal coil is different from the pitch of the proximal coil. In a refinement, the pitch of the distal coil is smaller than that of the proximal coil.

The fluid-removing tube may be manufactured from any material that is suitable for its application. As a non-limiting example, the material may be selected from the group consisting of rubbers, plastics, metals, or laminates thereof typically used in medical applications. The fluid-removing tube and the endotracheal tube may be made of the same or different materials. The relatively rigid construction of the fluid-removing tube may help reducing folding, kinking, or other deformation that may lead to blockage of the lumen.

Similarly, the fluid-removing tube may be of any dimension that is suitable for its application. The distal and proximal coils of the three-dimensional structure may have a helical radius that is slightly larger than the outer diameter of the endotracheal tube so that the coils may be securely and slidably mounted along the exterior surface of the endotracheal tube. The distal and proximal coils may have different pitches.

The fluid-removing tube may have an inner/outer diameter combination that enables a relatively rigid tube construction without limiting the flow of the accumulated fluid therein. The distal fluid port and the proximal fluid port, when provided as transverse openings on the tubular wall of the fluid-removing tube, may have diameters similar to the inner diameter of the tube.

In use, the disclosed apparatus may be mounted on an endotracheal tube that is already placed in the respiratory tract of a patient. To mount the disclosed apparatus, the distal coil of the fluid-removing tube may be first mounted on a proximal end of the endotracheal tube so that the distal coil wraps around the exterior tubular surface of the proximal end. Then, in a distal sliding and rotating movement, the distal coil is advanced toward the inflatable cuff until after the distal fluid port of the fluid-removing tube is in communication with the fluid accumulated between the endotracheal tube and the respiratory tract and above the inflatable cuff. Alternatively, the disclosed apparatus may be pre-mounted on an endotracheal tube before the endotracheal tube is inserted into the respiratory tract.

After the distal fluid port is in communication with the accumulated fluid, the accumulated fluid may be removed from the respiratory tract through the lumen by applying a pressure differential between the proximal fluid port and the distal fluid port. The pressure differential between the proximal fluid port and distal fluid port may be applied through one of many mechanisms known in the field of fluid dynamics. For example, the pressure difference may be effectuated by applying a suction force at the proximal fluid port or by a siphon effect of positioning the proximal fluid port lower than the distal fluid port.

In one embodiment, the application of pressure differential may be continuous, in which case the accumulated fluid is constantly removed by the disclosed apparatus. In another embodiment, the pressure differential may be applied as needed or at predetermined time intervals. In yet another embodiment, the application of the pressure differential may be synchronized to certain physiological activity of the patient.

Other advantages and features will be apparent from the following detailed description when read in conjunction with the attached drawings. It will also be noted here and elsewhere that the valve elements and assemblies disclosed herein may be suitably modified to be used in a wide variety of industrial operations by one of ordinary skill in the art without undue experimentation.

### BRIEF DESCRIPTION OF THE DRAWINGS

For a more complete understanding of the disclosed fluid-removing apparatus, reference should be made to the embodiments illustrated in greater detail in the accompanying drawings, wherein:

FIG. 1 is a perspective view of an endotracheal tube that may be used with the disclosed apparatus;

FIG. 2A is a perspective view of one embodiment of the disclosed apparatus;

FIG. 2B is a perspective view of another embodiment of the disclosed apparatus, particularly illustrating the optional proximal coil having the same pitch as the distal coil;

FIG. 2C is a perspective view of another embodiment of the disclosed apparatus, particularly illustrating the optional proximal coil having a different pitch from the distal coil;

FIG. 3 is a perspective view of the embodiment of the disclosed apparatus as shown in FIG. 2B, particularly illustrating the mounting of the distal coil onto the proximal end of an endotracheal tube;

FIG. 4 is a perspective view of the embodiment of the disclosed apparatus as shown in FIG. 2B, particularly illustrating the advancement of the distal coil toward the inflatable cuff; and

FIG. 5 is a perspective view of the embodiment of the disclosed apparatus as shown in FIG. 2B, particularly illustrating the removal of the accumulated fluid through the lumen of the fluid-removing tube.

It should be understood that the drawings are not necessarily to scale and that the disclosed embodiments are sometimes illustrated diagrammatically and in partial views. In certain instances, details which are not necessary for an understanding of the disclosed apparatus or which render other details difficult to perceive may have been omitted. It should be understood, of course, that this disclosure is not limited to the particular embodiments illustrated herein.

### DETAILED DESCRIPTION OF THE DISCLOSURE

This disclosure is generally directed toward a medical apparatus for removing fluid accumulated along a respiratory tract of a patient, and particularly around an endotracheal tube inserted therein. The disclosed apparatus may include a tube defining a lumen extending between a distal fluid port and a proximal fluid port thereon, wherein the tube forms a self-supported, three-dimensional structure. The three-dimensional structure may include a distal coil adapted to be slidably mounted on the exterior surface of the endotracheal tube. The three-dimensional structure may also include an optional proximal coil.

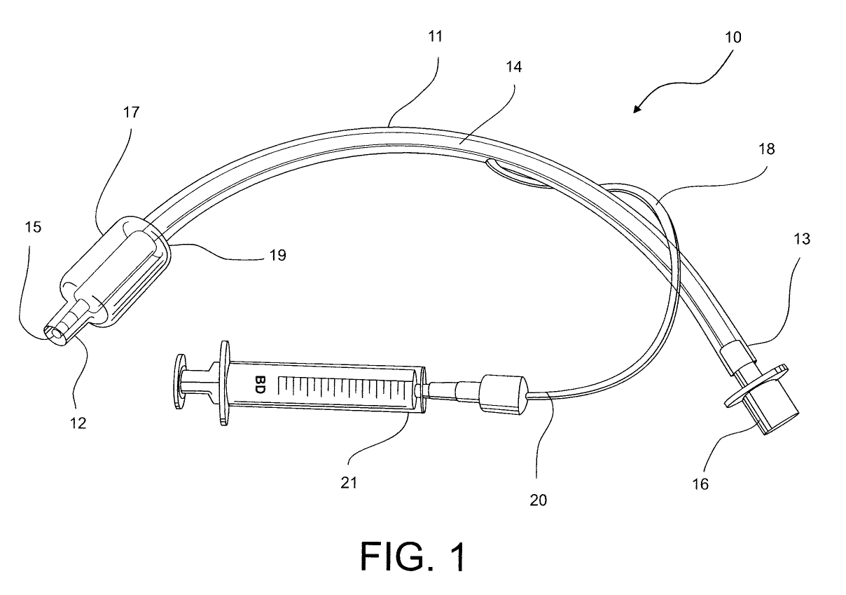

A conventional endotracheal tube for keeping an airway open is illustrated in FIG. 1. The endotracheal tube 10 may include a distal end 12, a proximal end 13, and a tubular wall 11 extending therebetween. The tubular wall 11 defines a lumen 14 extending between a distal opening 15 and a proximal opening 16. The tubular wall 11 further includes an inflatable cuff 17 disposed close to the distal end 12. The endotracheal tube 10 also includes an inflation tube 18 having a distal end 19 in communication with the inflatable cuff 17, and a proximal end 20 adapted to be connected to a fluid or gas infusion device 21, such as a syringe.

In use, the endotracheal tube 10 is inserted into the respiratory tract of a patient with the cuff 17 deflated. After the endotracheal tube 10 is properly positioned within the respiratory tract, fluid or gas is infused through the inflation tube 18 to inflate the cuff 17 so that the cuff 17 circumferentially engages the respiratory tract. The proximal end 13 of the endotracheal tube 10 may be connected to a medical apparatus such as a ventilator, or it may be left open to atmosphere so that the patient can breathe on his or her own.

Depending on the condition of the patient and the duration of the medical treatment, the endotracheal tube 10 may need to be placed in the respiratory tract for a prolonged period of time, during which body secretions such as saliva and mucus may accumulate around the endotracheal tube 10 above the inflated cuff 17. The accumulated fluid may serve as a breeding ground for harmful microorganisms which, if leaked through the cuff 17 into the lower respiratory tract or lungs, may cause serious medical problems such as inflammation or pneumonia.

Although the inflated cuff 17 may provide a basic seal along the respiratory tract it circumferentially engages, the seal may not be fluid-tight to prevent the accumulated fluid from leaking through. In particular, the exterior surface of the cuff 17 may not be perfect and free from creases and grooves. Further, movement of the patient and thus the respiratory tract may cause momentary deformation to the cuff 17. As a result, small channels may be form between the cuff 17 and the respiratory tract, through which the accumulated fluid may leak into the lower respiratory tract or lungs. Thus, a medical apparatus that continuously or periodically removes the accumulated fluid from the respiratory tract may help to minimize or significantly reduce the risk of aforementioned medical problems.

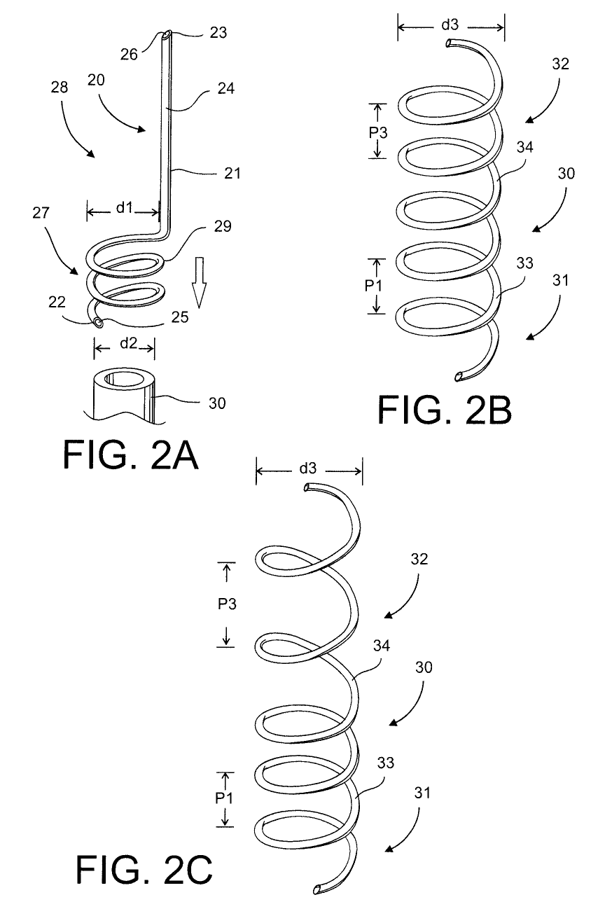

FIGS. 2A-2C illustrate some embodiments of the apparatus for removing fluid accumulated around an endotracheal tube contemplated by this disclosure. As shown in FIG. 2A, the apparatus 20 may include a fluid-removing tube 21 having a distal end 22 and a proximal end 23. The tube 21 defines a lumen 24 extending between a distal fluid port 25 and a proximal fluid port 26. In the embodiment illustrated in FIG. 2A, the distal and proximal ends (22, 23) of the tube 21 are both open, in which case the distal and proximal fluid ports (25, 26) are simply provided as the distal and proximal openings of the tube 21.

Alternatively, the distal end 22 of the fluid-removing tube 21 may be closed, in which case the distal fluid port 25 may be provided as a transverse opening on the tubular wall close to the distal end 22 of the fluid-removing tube 21. Similarly, the proximal end 23 of the fluid-removing tube 21 may also be closed, in which case the proximal fluid port 26 may be provided as a transverse opening on the tubular wall close to the proximal end 23 of the fluid-removing tube 21. Other suitable locations for the fluid ports (25, 26) should be obvious to one of ordinary skill in the art in view of this disclosure and should be considered as within the scope of this disclosure.

The fluid-removing tube 21 may have a suitable inner/outer diameter combination that enables a relatively rigid tube construction. Moreover, as the fluid accumulated around the endotracheal tube may be thick and/or tacky, the fluid-removing tube 21 may have an inner diameter large enough to enable adequate flow of the accumulated fluid therein. Furthermore, the distal and proximal fluid ports (25, 26), when provided as transverse openings on the tubular wall of the fluid-removing tube 21, may have diameters equal to or larger than the inner diameter of the tube 21 so that the fluid ports (25, 26) do not unduly restrict the flow of the accumulated fluid in and out of the fluid-removing tube 21.

The fluid-removing tube 21 may be manufactured from any material that is suitable for its application. As a non-limiting example, the material may be selected from the group consisting of rubbers, plastics, metals, or laminates thereof typically used in medical tubing. In one embodiment, the fluid-removing tube is manufactured from nylon. The fluid-removing tube and the endotracheal tube may be made of the same or different materials. The relatively rigid construction of the fluid-removing tube 21 may help reduce folding, kinking, or other deformation that may lead to blockage of the lumen 24.

To that end, the fluid-removing tube may have sufficient rigidity to form a self-supported, three-dimensional structure, as illustrated in FIGS. 2A-2C. In particular, the three-dimensional structure may include a distal portion connected to a proximal portion, wherein the distal portion includes a distal coil adapted to be slidably mounted along the exterior surface of an endotracheal tube.

Turning to FIG. 2A, the three-dimensional structure of the fluid-removing tube 20 includes a distal portion 27 and a proximal portion 28. The distal portion includes a distal coil 29. The helical radius (d₁) of the distal coil 29 may be larger than the outer diameter (d₂) of the endotracheal tube 30 so that the distal coil 29 can be slidably mounted along the exterior surface of the endotracheal tube 30. As a non-limiting example, the ratio of d₁/d₂ may range from about 1.001 to about 1.500, more preferably from about 1.001 to about 1.200. Although the proximal portion 28 of the three-dimensional structure is shown in FIG. 2A as having a simple linear design, it may also include bends, curves, or other shapes known in the art so long as the overall shape of the proximal end 28 does not interfere with the sliding movement of the distal coil along the exterior surface of the endotracheal tube 30.

In the embodiments illustrated in FIGS. 2B-2C, the three-dimensional structure of the fluid-removing tube 30 includes distal and proximal portions (31, 32). The distal portion 31 includes a distal coil 33. The proximal portion 32 may optionally include a proximal coil 34. The helical radius (d₃) of the proximal coil 34 may be larger than the outer diameter (d₂) of the endotracheal tube 30 so that the proximal coil 34 can be slidably mounted along the exterior surface of the endotracheal tube 30. As a non-limiting example, the ratio of d₃/d₂ may range from about 1.001 to about 1.500, more preferably from about 1.001 to about 1.200.

In the embodiment illustrated in FIG. 2B, the pitch of the proximal coil (p₃) may be identical to the pitch of the distal coil (p₁). However, the pitches of the proximal and distal coils (p₃, p₁) may also be different. As illustrated in FIG. 2C, the pitch of the proximal coil (p₃) may be greater than the pitch of the distal coil (p₁). In one embodiment, the pitch of the distal end may be from about 0.5 cm to about 2 cm.

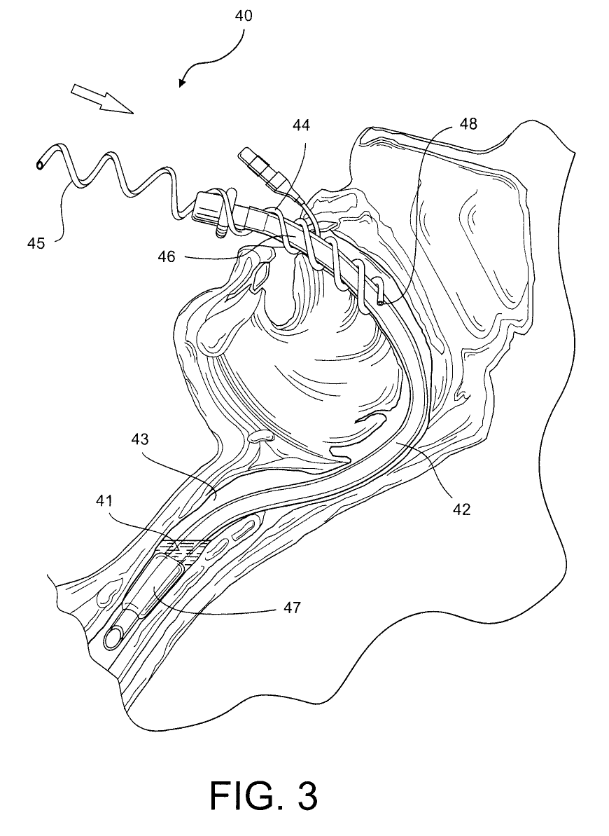

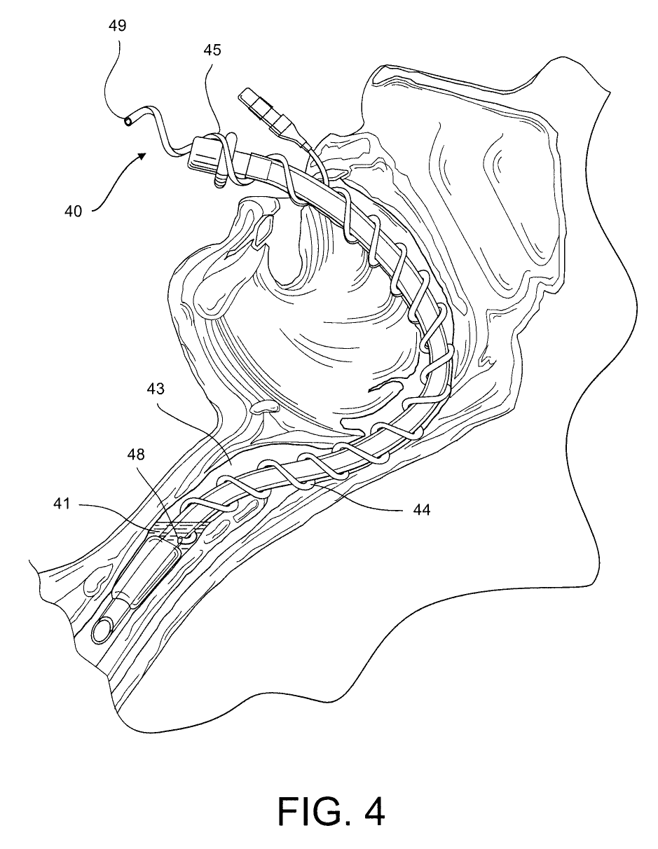

Turning now to FIGS. 3-5, which illustrate the use of the disclosed apparatus 40 to remove fluid 41 accumulated around the endotracheal tube 42 and the respiratory tract 43 of a patient. As shown in FIG. 3, the distal coil 44 of the fluid-removing tube 45 may be first mounted on the proximal end 46 of the endotracheal tube 42 so that the distal coil 44 wraps around the exterior tubular surface of the proximal end 46. After being securely mounted on the proximal end 46, the distal coil 44 is advanced, in a distally sliding and rotating movement guided by the endotracheal tube 42 as indicated by the arrow in FIG. 3, toward the inflatable cuff 47 of the endotracheal tube 42 until after the distal fluid port 48 is in communication with the accumulated fluid 41. In one embodiment, the distal coil 44 is advanced until it engages the inflatable cuff 47. In another embodiment, the distal coil 44 is advanced until an indicium provided on the proximal portion of the fluid-removing tube 45 is aligned with a predetermined mark provided on the endotracheal tube 42. Although the endotracheal tube 42 is shown in FIGS. 3-4 as prepositioned in the respiratory tract 43 before the mounting of the distal coil 44, the disclosed apparatus may also be pre-mounted on the endotracheal tube 42 and inserted with the endotracheal tube 42 into the respiratory tract 43.

Because the disclosed apparatus 40 is relatively rigid, the mounting, sliding, and rotating of the distal coil 44 along the endotracheal tube 42 is unlikely to cause any folding, kinking or other deformation to the apparatus 40. As a result, the fluid-removing tube 45 may be less susceptible to clogging and/or damage than conventional fluid-removing devices that use flexible tubes. On the other hand, the distal coil design may minimize or prevent trauma to the tissues along the respiratory tract caused by the sliding movement of a relatively rigid tube. Toward that end the distal tip of the coil 44 may be formed from a soft or malleable material.

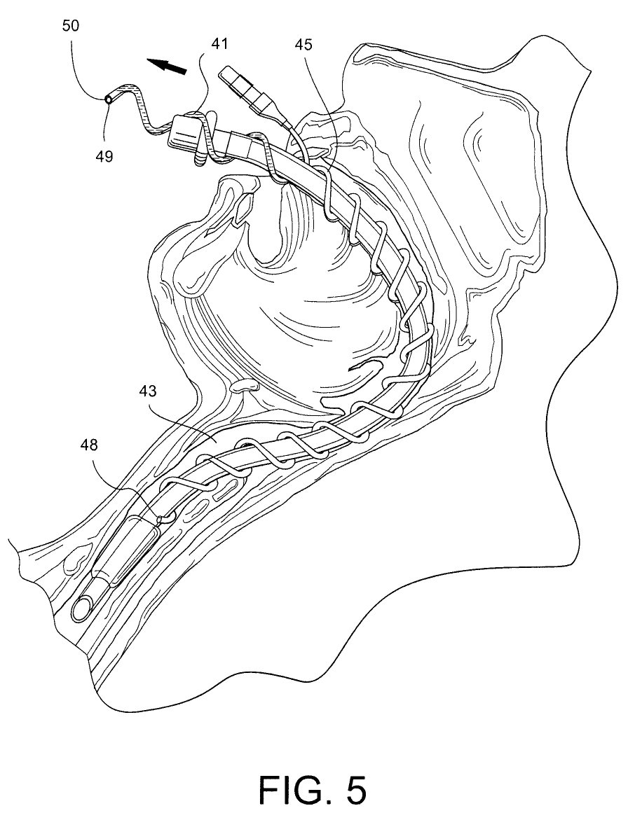

After the distal fluid port 48 is in communication with the accumulated fluid 41 as illustrated in FIG. 4, the accumulated fluid 41 may be removed from the respiratory tract 43 by applying a pressure differential between the proximal fluid port 49 and the distal fluid port 48. More specifically, if the pressure at the proximal fluid port 49 is lower than the pressure at the distal fluid port 48, the accumulate fluid 41 will enter the fluid-removing tube 45 through the distal fluid port 48 and exit the tube 45 through the proximal fluid port 49, as illustrated by the arrow in FIG. 5. Although the embodiment illustrated in FIGS. 3-5 uses the fluid-removing tube shown in FIG. 2B, it is to be understood that the disclosed apparatus shown in FIGS. 2A and 2C may also be used in a similar fashion.

The pressure differential between the proximal fluid port 49 and distal fluid port 48 may be applied through one of many mechanisms known in the field of fluid transportation. For example, the pressure differential may be effectuated by connecting the proximal fluid port 49 with a suction device 50. The suction device may be a medical aspirator, a syringe, a pump, or any other manual or automatic device used in the medical field to supply a suction force.

It is to be understood, however, that the use of the suction device 50 may be optional. For example, the proximal fluid port 49 may be connected to a central vacuum system commonly installed in medical institutions. In another alternative embodiment, the proximal fluid port 49 may be positioned lower than the distal fluid port 48, in which case the accumulated fluid 41 may be removed from the respiratory tract 43 simply through a siphon effect.

In one embodiment, the application of pressure differential may be continuous, in which case the accumulated fluid is constantly removed by the disclosed apparatus. In another embodiment, the pressure differential may be applied as needed or at predetermined time intervals. In yet another embodiment, the application of the pressure differential may be synchronized to certain physiological activity of the patient, such as breathing, etc.

In general, when properly installed, the position of the disclosed apparatus 40 is left undisturbed until the endotracheal tube 42 is no longer needed, in which case the apparatus 40 may be removed from the respiratory tract 43, either separately or together with the endotracheal tube 42. If the disclosed apparatus 40 is damaged or clogged, the apparatus 40 may be conveniently removed from the respiratory tract 43 by a proximal sliding and rotating movement without the need to remove the endotracheal tube 42, which causes less discomfort to the patient than an apparatus that integrates the fluid-removing tube with the endotracheal tube.

Numerous modifications and variations of the disclosed apparatus are possible in light of the above disclosure. For example, the apparatus may be used or modified to remove fluid from body cavities other than a respiratory tract without undue experimentation. It is therefore to be understood that, within the scope of the appended claims, the invention may be practiced otherwise than as specifically described herein. While only certain embodiments have been set forth, alternatives and modifications will be apparent from the above description to those skilled in the art. These and other alternatives are considered equivalents and within the spirit and scope of this disclosure and the appended claims.

What is claimed is:

1. A fluid-removing system, comprising:
an endotracheal tube adapted to be inserted in a trachea, the endotracheal tube including an inflatable cuff at a distal end; and
a suction tube defining a lumen extending between a distal fluid port and a proximal fluid port thereon, the tube forming a self-supported, three-dimensional structure, the three-dimensional structure having a distal portion and a proximal portion, the distal portion having a distal coil.

2. The system of claim 1, wherein the distal fluid port is a distal opening of the tube.

3. The system of claim 1, wherein the proximal fluid port is a proximal opening of the tube.

4. The system of claim 1, wherein the proximal portion comprises a proximal coil.

5. The system of claim 4, wherein the pitches of the distal and proximal coils are different from each other.

6. The system of claim 5, wherein the pitch of the distal coil is smaller than the pitch of the proximal coil.

7. The system of claim 1, wherein the pitch of the distal coil is from about 0.5 cm to about 2 cm.

8. A fluid-removing apparatus for removing fluid accumulated around an endotracheal tube, the apparatus comprising:
a tube defining a lumen extending between a distal fluid port and a proximal fluid port thereon; and,
a distal coil provided at the distal fluid point adapted to be slidably mounted along the exterior surface of the endotracheal tube.

9. The apparatus of claim 8, wherein the distal fluid port is a distal opening of the tube.

10. The apparatus of claim 8, wherein the proximal fluid port is a proximal opening of the tube.

11. The apparatus of claim 8, wherein a helical radius of the distal coil is larger than an outer diameter of the endotracheal tube.

12. The apparatus of claim 8, wherein the pitch of the distal coil is from about 0.5 cm to about 2 cm.

13. The apparatus of claim 8, wherein the apparatus further includes proximal coil adapted to be slidably mounted on the exterior surface of the endotracheal tube.

14. The apparatus of claim 13, wherein the pitch of the proximal coil is greater than the pitch of the distal coil.

15. A method for removing fluid accumulated between an endotracheal tube and a respiratory tract, comprising:
providing a fluid-removing tube defining a lumen extending between a distal fluid port and a proximal fluid port thereon, the fluid-removing tube forming a self-supported, three-dimensional structure comprising a distal coil;
mounting the distal coil along the exterior surface of the endotracheal tube;
distally advancing the distal coil along the respiratory tract until after the distal fluid port is in communication with the accumulated fluid; and
removing the accumulated fluid by applying a pressure differential between the distal fluid port and the proximal fluid port.

16. The method of claim 15, wherein the distal coil is distally advanced by rotating the tube fluid-removing tube around the endotracheal tube.

17. The method of claim 15, wherein the accumulated fluid is removed by connecting the proximal fluid port to a suction source.

18. The apparatus of claim 15, wherein a helical radius of the distal coil is larger than an outer diameter of the endotracheal tube.

19. The method of claim 15, wherein the three-dimensional structure further comprises a proximal coil adapted to be slidably mounted along the exterior surface of the endotracheal tube.

20. The method of claim 19, wherein the pitch of the proximal coil is greater than the pitch of the distal coil.
